(19) United States
(12) Patent Application Publication
(10) Pub. No.: US 2007/0042971 A1
(43) Pub. Date: Feb. 22, 2007

(54) Crystalline compound of 4'-demethyl-4'-phosphate-2", 3"-bispentafluorophenoxy-acetyl-4", 6"-ethylidene-beta-d-epipodophyllotoxin glucoside either in its free form or solvated with ethanol

(75) Inventors: Yves Guminski, Lagarrigue (FR); Thierry Imbert, Viviers-Les-Montagnes (FR); Anna Kruczynski, Castres (FR)

Correspondence Address: BIRCH STEWART KOLASCH & BIRCH, PO BOX 747, FALLS CHURCH, VA, 22040-0747, US

(73) Assignee: Pierre Fabre Medicament, Boulogne-Billancourt (FR)

(21) Appl. No.: 10/572,061

(22) PCT Filed: Sep. 16, 2004
(86) PCT No.: PCT/FR04/02349

§ 371 Date: Mar. 15, 2006

(30) Foreign Application Priority Data
Sep. 16, 2003 (FR) 03/10.836

(51) Int. Cl. A61K 31/7048; C07H 15/02

(52) U.S. Cl. 514/27; 536/17.1

(57) ABSTRACT

The invention relates to a crystalline compound of 4′-demethyl-4′-phosphate-2″,3″-bispentafluorophenoxyacetyl-4″,6″-ethylidene-β-D-epipodophyllotoxin glucoside in its free form or solvated with ethanol, advantageously provided in the form of a hemiethanolate solvate. The invention also relates to a method for preparing these compounds and to their use as an anticancer drug.

4′-demethylepipodophyllotoxin 2″,3″-bispentafluoro-phenoxyacetyl-4″,6″-ethylidene-β-D-glucoside 4′-phosphate in N-methyl-D-glucamine disalt form, according to formula 1, is known by virtue of its structure, the method for preparing it and its anticancer activity (WO 96/12727). Another method for preparing this compound is also described in patent FR 2 791 682.

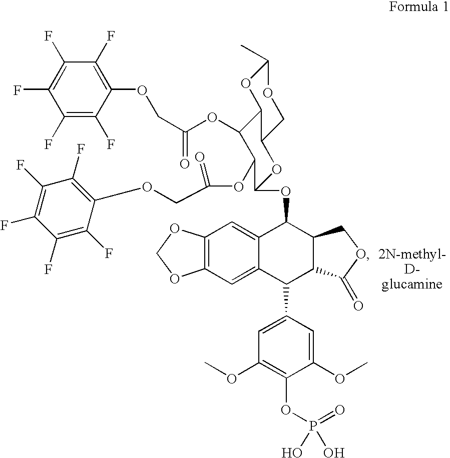

This compound of formula 1 has a particularly considerable anticancer activity. Its antitumor activity has been demonstrated in vivo. In particular, it allows a long survival in mice in which the P388 leukemia model has been implanted (British Journal of Cancer (2000), 83(11), 1516-1524). Furthermore, the potency of the inhibition of topoisomerases 1 and 2 activity in vitro has been shown with this compound (Biochem. Pharmacol. (2000), 59, 807).

This compound 1 is stable under the usual conditions of neutral and acid pH and of temperature. However, if it is desired to conserve it over a long period of time, it is necessary to conserve it in a dry place in a hermetic bell bottle at −20° C. On the other hand, if it is conserved at 4° C. and, a fortiori, at ambient temperature, this compound degrades over time. It cannot therefore be handled in a satisfactory manner so as to be used in an anticancer treatment protocol in the hospital environment. It must therefore be prepared at the time of use, which is a handicap.

The chemical precursor of this compound is the free phosphate derivative, of formula 2: 4′-demethylepipodophyllotoxin 2″,3″-bispentafluoro-phenoxyacetyl-4″,6″-ethylidene-β-D-glucoside 4′-phosphate.

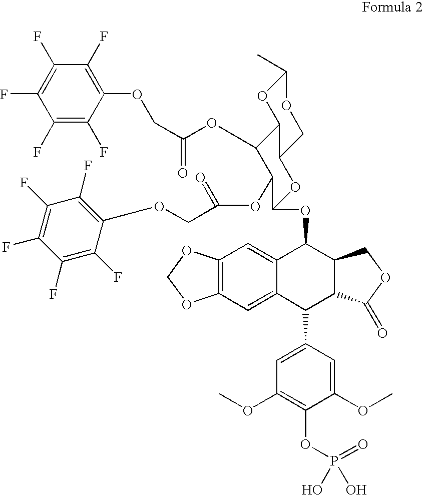

This compound is not isolated and is used directly in amorphous form in the preparation of its N-methyl-D-glucamine salt, of formula 1 (WO 96/12727). This free phosphate derivative of formula 2, once isolated, is in a hygroscopic and relatively unstable amorphous form. It is then necessary to form the N-methyl-D-glucamine salt rapidly, under anhydrous conditions, and to conserve it in the cold, or to use it rapidly. This derivative of formula 2 in amorphous form cannot therefore itself be stored and handled under good conditions on an industrial level. Furthermore, it has been observed that the compound of formula 2, in amorphous form, does not exhibit the important biological properties of the compound of formula 1. A problem therefore remains to be solved, that of obtaining the compound of formula 2 in crystalline form.

It is conventionally known that the crystallization of an amorphous compound can pose very great difficulties, and the obtaining of the first crystals is always problematic.

Subsequently, the crystallization occurs more readily by seeding.

A solvate with 2 ethanol molecules has been described in patent EP 537555 for the compound etopophos, which is the prodrug of etoposide. It was logical to hope to obtain crystallization of the compound of formula 2 by carrying out a procedure similar to the process described in that patent.

The method for obtaining the solvate, diethanolate, specified in that patent (treatment with ethanol with a cosolvant or water) is not suitable in our case.

There is a very substantial difference in solubility between etopophos and the product of formula 2.

Etopophos is water-soluble (100 mg/ml) whereas the compound of formula 2, which is amorphous, is not. The latter is soluble in ethanol and the addition of water recommended for obtaining the solvate of etopophos gives, in our case, a noncrystallizable gum.

Many trials have been carried out in the laboratory in order to try to crystallize the compound of formula 2. Alcohols such as methanol, ethanol, isopropyl alcohol or butanol have been used without success, as have other types of solvents, such as ethyl acetate, acetone, methyl ethyl ketone or dioxane. Only the use of ethers, such as ethyl ether or isopropyl ether, or alkanes, such as pentane, heptane or petroleum ether, provides a product which is a solid powder but is in amorphous form.

It has therefore been found, surprisingly, that the free phosphate of formula 2 has the property of forming, under specific conditions, a solvate with ½ molecule of ethanol so as to give its crystalline and stable hemiethanolate derivative, of formula 3, this derivative giving, after drying, the derivative of formula 2 in crystalline form. Furthermore, by seeding using the crystalline compound of formula 2 or 3, it has been possible to obtain crystalline solvates with ethanol other than the hemiethanolate solvate of 4′-demethylepipodophyllotoxin 2″,3″-bispentafluoro-phenoxyacetyl-4″,6″-ethylidene-β-D-glucoside 4′-phosphate.

The present invention therefore relates to the crystalline compound 4′-demethylepipodophyllotoxin 2″,3″-bispentafluorophenoxyacetyl-4″,6″-ethylidene-β-D-glucoside 4′-phosphate in free form of formula 2 below: 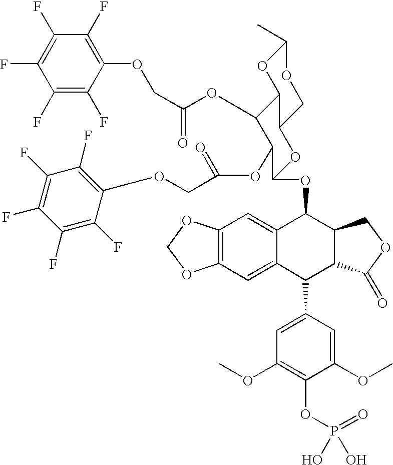
or in the form solvated with ethanol. Advantageously, the solvate with ethanol is a hemiethanolate solvate of formula 3 below:

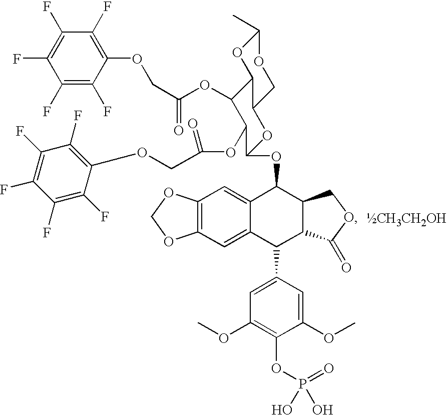

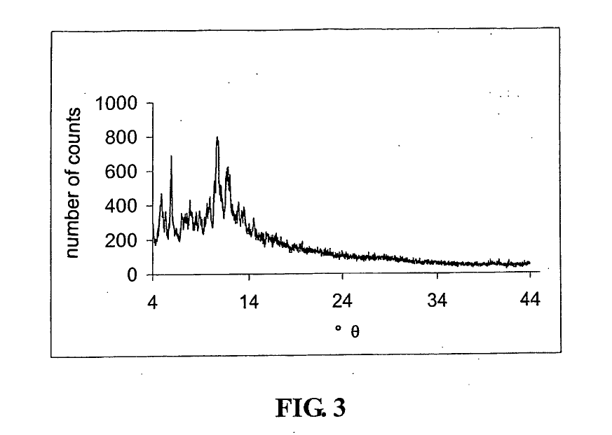

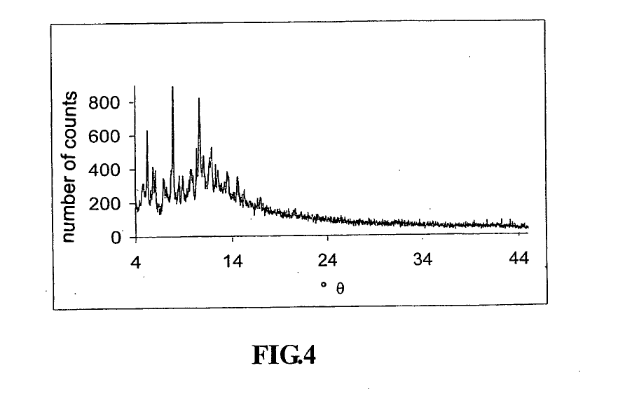

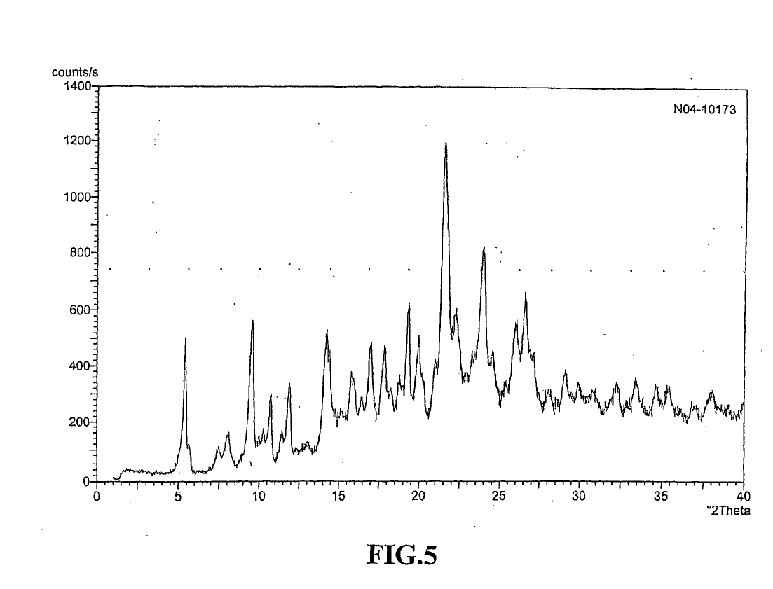

Advantageously, the X-ray diagram of the crystalline compound in free form of formula 2 corresponds substantially to that of FIG. 2, and that of the crystalline compound in the form of the hemiethanolate solvate of formula 3 corresponds substantially to that of FIG. 3. Advantageously, the X-ray diagram of a crystalline compound of 4′-demethylepipodophyllotoxin 2″,3″-bispentafluorophenoxyacetyl-4″,6″-ethylidene-β-D-glucoside 4′-phosphate in a form solvated with ethanol other than the hemiethanolate solvate of formula 3 corresponds substantially to that of FIG. 4.

Other subjects and advantages of the invention will become apparent to those skilled in the art from the detailed description below, and via references to the following illustrative drawings.

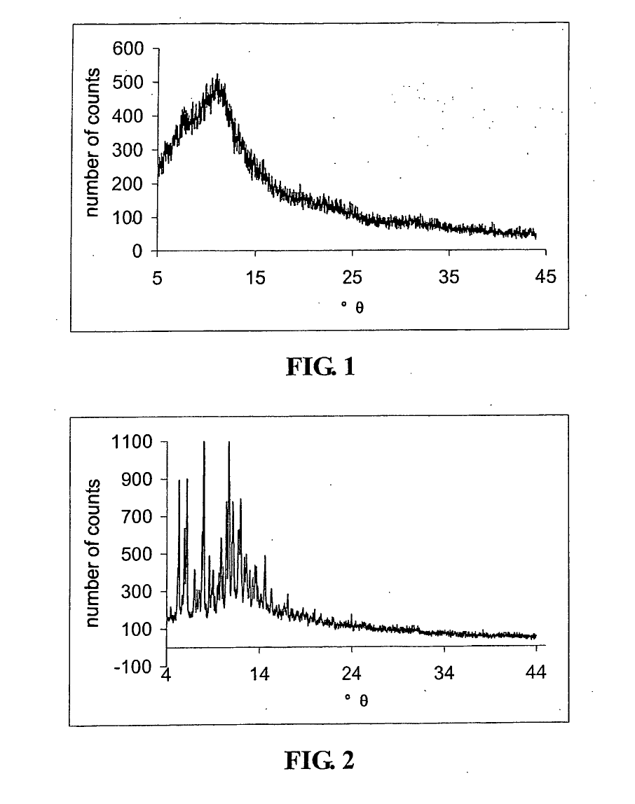

FIG. 1 represents the X-ray diagram of the compound of formula 2 in amorphous form.

FIGS. 2 and 6 represent the X-ray diagram of the compound of formula 2 in crystalline form.

FIG. 3 represents the X-ray diagram of the compound of formula 3 in crystalline form.

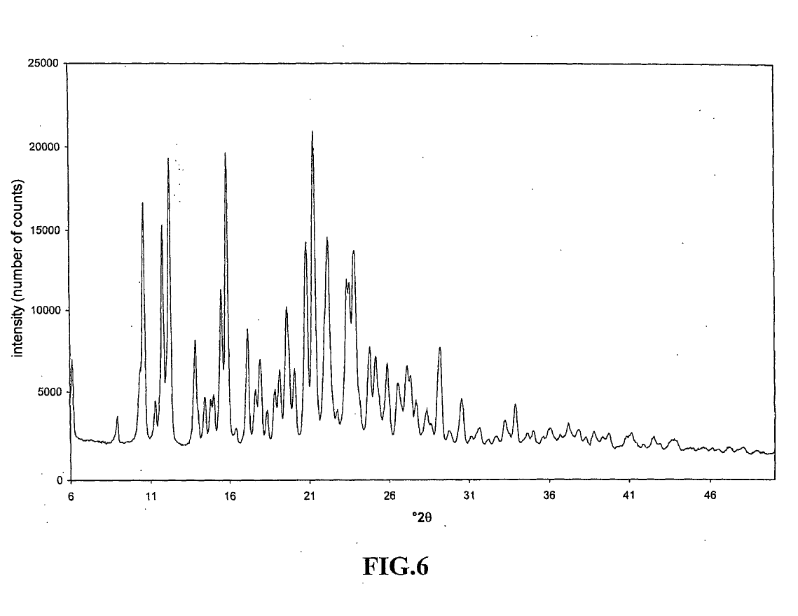

FIGS. 4 and 5 represent the X-ray diagram of the crystalline compound of 4′-demethylepipodophyllotoxin 2″,3″-bispentafluorophenoxyacetyl-4″,6″-ethylidene-β-D-glucoside 4′-phosphate in a form solvated with ethanol other than the hemiethanolate solvate of formula 3.

The present invention also relates to a method for preparing the crystalline compound in the form of the hemiethanolate solvate of formula 3, which comprises the step consisting in dissolving the compound of formula 2 in amorphous form in anhydrous ethanol in the presence of ultrasound.

Advantageously, the ultrasound is applied for at least 20 minutes.

It also relates to the method for preparing the crystalline compound in free form of formula 2, which comprises the step consisting in drying the crystalline compound in the form of the hemiethanolate solvate of formula 3, advantageously under vacuum.

In fact, treatment of the free phosphate of formula 2 in solution in anhydrous ethanol and in the presence of ultrasound, advantageously for 20 min., promotes the appearance of the crystalline form. It forms a solvate with half a molecule of ethanol for crystallization. This phenomenon does not appear to be general because comparative trials with methanol were carried out. No crystallization then appeared. Similarly, equivalent conditions were applied to the various compounds of the family of the product of formula 2, for example the products having a phenoxyacetate, 4-trifluoromethoxyphenoxyacetate or 4-methylphenoxy-acetate chain instead and in place of the pentafluorophenoxyacetate chain, which products are mentioned in patent application WO 96/12727. No crystallization occurs with these similar structures. Only the compound of formula 2 having the pentafluorophenoxyacetate chain has the particularity of forming crystals through the treatment mentioned above.

After several recrystallizations, this compound still keeps the same ethanol stoichiometry, i.e. ½ mol of solvent per mole of the compound, even after drying under vacuum at ambient temperature. At this stage, a solvate is obtained, the hemiethanolate of formula 3.

Furthermore, it became apparent that more extensive drying under vacuum at 40° C. for 10 days then makes it possible to eliminate with difficulty but completely the ethanol. It is thus necessary to go through a crystalline form as solvate before obtaining a crystalline form without solvent. This compound cannot be obtained directly.

It is clearly understood that the major difficulty in crystallization lies in obtaining the first crystal, and that other crystallization techniques can be involved and can result in crystallization of future batches by seeding with already formed crystals. Thus, the present invention also relates to a method for preparing the crystalline compound of 4′-demethylepipodophyllotoxin 2″,3″-bispentafluoro-phenoxyacetyl-4″,6″-ethylidene-β-D-glucoside 4′-phosphate in the form solvated with ethanol, characterized in that it comprises the following successive steps:
a) addition of ethanol to the compound 2 in amorphous form,
b) concentration of the solution,
c) crystallization of the 4′-demethylepipodophyllotoxin 2″,3″-bispentafluoro-phenoxyacetyl-4″,6″-ethylidene-β-D-glucoside 4′-phosphate in the form solvated with ethanol, by cooling the concentrated solution at 10° C. for 1.5 h and adding an initiator consisting of a crystal of the compound of formula 2 in free form or of the compound of formula 3 in the form of the hemiethanolate solvate,
d) filtration at 10° C. and rinsing with ethanol,
e) drying of the product obtained.

Step (a) consisting of concentration of the solution is advantageously carried out by evaporation of approximately 50% of the solution.

Advantageously, if the crystalline product obtained by means of the method according to the present invention is not in comformity, a second recrystallization is carried out by resolubilizing in acetone, by preconcentrating the solution obtained, advantageously by evaporating 75% of the solution, and then by repeating steps (a) to (e).

Advantageously, step (e) consisting of drying of the product is carried out in an oven, under vacuum and at 40° C.

It has also become apparent that extensive drying under vacuum (advantageously at 40° C. for 10 days) then makes it possible to eliminate with difficulty but completely the ethanol. A crystalline form without solvent is thus obtained, i.e. the crystalline compound of formula 2 in free form.

The present application also relates to a medicinal product comprising a crystalline compound according to the present application (advantageously of formula 2 or 3).

Advantageously, this medicinal product is for use in anticancer treatment, advantageously for liquid tumors or solid tumors, in particular small cell lung cancer, embryonic tumors, neuroblastomas, kidney cancer, pediatric tumors, Hodgkin's and nonhodgkins lymphomas, acute leukemias, placental choriocarcinomas, and mammary adenocarcinomas.

In particular, this medicinal product is intended to increase the therapeutic efficacy of topoisomerase 1 or 2 inhibitor compounds for the treatment of tumors that are refractory to the usual therapies, such as colorectal cancers, melanomas, gliomas, prostate cancer and breast cancer.

In an advantageous embodiment, the present medicinal product is an antitumor agent for use in treating any type of tumor, whether liquid tumors or solid tumors. Advantageously, the medicinal product according to the present invention is in a form intended for oral administration, advantageously in the form of gelatin capsules, of capsules or of tablets, or parenteral administration, advantageously at the dosage per 24 h of 5 to 400 mg/m² for oral administration and of 2 to 200 mg/m² for administration by injection.

The medicinal product according to the present invention can be administered to patients as a first line treatment or after one or more treatments by surgery, radiotherapy or chemotherapy. It can be administered alone or in combination with other chemotherapies. This medicinal product can therefore also comprise another anticancer agent.

The following examples are given by way of nonlimiting indication.

Method of Production

### EXAMPLE 1

### Preparation of the hemiethanolate of 4′-demethylepipodophyllotoxin 2″,3″-bispentafluoro-phenoxyacetyl-4″,6″-ethylidene-β-D-glucoside 4′-phosphate from 4′-demethylepipodophyllotoxin 2″,3″-bispentafluorophenoxyacetyl-4″,6″-ethylidene-β-D-glucoside 4′-dibenzylphosphate in amorphous form

The free phosphate of formula 2 in amorphous form is obtained either by the method described in patent application WO 96/12727 in example 22, or according to that described in example 8 of patent FR 2 791 682. For example: 15 g (11.6 mmol) of 4′-demethylepipodophyllotoxin 2″,3″-bispentafluoro-phenoxyacetyl-4″,6″ethylidene-β-D-glucoside 4′-dibenzylphosphate are placed, in order to be hydrogenated, in 200 ml of a mixture of ethyl acetate-ethanol (⅓ ), under a hydrogen atmosphere, at normal pressure, with 1.2 g of palladium-on-charcoal at 10%, for 30 min with stirring. The reaction medium is filtered, and the filtrate is evaporated and triturated from isopropyl ether so as to obtain an amorphous powder of formula 2.

Melting point (Electrothermal 9300 capillary melting point apparatus): ˜138° C. (softening). Analytical purity HPLC: 99.5%. TLC: Rf 0.5, solvent: dichloromethane-methanol-acetic acid 90/10/5.

X-ray diagram: FIG. 1

Preparation of the Hemiethanolate:

10 g of the amorphous form obtained above are dissolved in 500 ml of ethanol. This solution is placed in an ultrasonic bath (Bioblock-Scientific type T 700, frequency 35 kHz) for 20 min, at 30° C. A thick precipitate appears, which is subsequently filtered off and dried under vacuum. 7 g of crystals are obtained. Melting point: 161° C. The NMR spectrum shows the presence of ½ mol of ethanol. These crystals are recrystallized a second time and then a third time from 130 ml of ethanol at reflux, and then are left to cool slowly for 12 h. After filtration and drying under vacuum, crystals are obtained.

Melting point 165-166° C. The NMR spectrum shows once again the presence of ½ mol of ethanol.

X-ray diagram: FIG. 3.

### EXAMPLE 2

### Preparation of 4′-demethylepipodophyllotoxin 2″,3″-bispentafluorophenoxyacetyl-4″,6″-ethylidene-β-D-glucoside 4′-phosphate in crystalline form from the hemiethanolate of 4′-demethylepipodophyllotoxin 2″,3″-bispentafluorophenoxyacetyl-4″,6″-ethylidene-β-D-glucoside 4′-phosphate in crystalline form

An aliquot of the crystals obtained above is placed, as a thin layer, in a heated desiccator at 40° C. for 10 days. The NMR spectrum shows the loss of ethanol. Melting point 169° C.

X-ray diagram: FIG. 2

### EXAMPLE 3

### Preparation of a crystalline solvate with ethanol of 4′-demethylepipodophyllotoxin 2″,3″-bispentafluoro-phenoxyacetyl-4″,6″-ethylidene-β-D-glucoside 4′-phosphate, other than the hemiethanolate

The phosphate of formula 2 in amorphous form is obtained either by the method described in patent application WO 96/12727 in example 22, or according to that described in example 8 of patent FR 2 791 682.

The crystallization steps are subsequently as follows:

addition of EtOH: approximately one volume relative to the initial volume before concentration,

concentration of the solution: evaporation of approximately 50% of the solution,

cooling of the solution to 10° C. and maintaining of this solution at 10° C. for 1.5 hours while at the same time seeding with a crystal of the compound obtained according to example 1 or 2,

filtration at 10° C. and rinsing with ethanol of the crystalline product obtained (if the product is not in conformity, a second recrystallization is carried out by resolubilizing in acetone and by preconcentrating, and then by repeating the steps indicated above),

drying of the product in an oven: under vacuum and at 40° C.

A crystalline solvate with ethanol is obtained.

X-ray diagram: FIGS. 4 and 5.

HPLC purity: 99.1%.

Ethanol content: 1.4%.

Water content: less than 1%.

### EXAMPLE 4

### Preparation of 4′-demethylepipodophyllotoxin 2″,3″-bispentafluorophenoxyacetyl-4″,6″-ethylidene-β-D-glucoside 4′-phosphate in crystalline form from the solvate with ethanol of 4-demethylepipodophyllotoxin 2″,3″-bispentafluorophenoxyacetyl-4″,6″-ethylidene-β-D-glucoside 4′-phosphate in crystalline form (example 3)

12 g of the crystals obtained above (example 3) are placed in a large crystallizing basin inside a vacuum bell jar with a heating platform. The vacuum is applied and the temperature is fixed at 40° C.

The crystals are dried for 10 days. At the end of these 10 days, the ethanol has completely evaporated.

An X-ray diffractogram was effected, showing complete crystallinity (FIG. 6).

### EXAMPLE 5

### Crystallization trials for 4′-demethylepipodophyllotoxin 2″,3″-bisphenoxyacetyl-4″,6″-ethylidene-β-D-glucoside 4′-phosphate, for 4′-demethylepipodophyllotoxin 2″,3″-bis(4-trifluoromethoxyphenoxyacetyl)-4″,6″-ethylidene-β-D-glucoside 4′-phosphate and for 4′-demethylepipodophyllotoxin 2″,3″-bis(4-methylphenoxyacetyl)-4″,6″-ethylidene-β-D-glucoside 4′-phosphate

The 4′-demethylepipodophyllotoxin 2″,3″-bisphenoxy-acetyl-4″,6″-ethylidene-β-D-glucoside 4′-phosphate, the 4′-demethylepipodophyllotoxin 2″,3″-bis(4-trifluoromethoxyphenoxyacetyl)-4″,6″-ethylidene-β-D-glucoside 4′-phosphate and the 4′-demethyl-epipodophyllotoxin 2″,3″-bis(4-methylphenoxyacetyl)-4″,6″-ethylidene-β-D-glucoside 4′-phosphate, in amorphous form, are obtained according to the method described in patent application WO 96/12727.

100 mg of these compounds are dissolved in 5 ml of ethanol at ambient temperature. These various solutions are placed in the ultrasonic bath for 20 min. No crystallization appears even after trituration of the walls, or by allowing to stand for several days, according to the usual methods for initiating crystallization.

Study of the Crystalline Form

X-ray diffraction studies on the various forms obtained above (formula 2 in amorphous form, in crystalline form, formula 3 in crystalline form and other crystalline compound in the form solvated with ethanol) and also thermomicroscopy studies were carried out on several samples in order to demonstrate the existence of the crystalline form.

X-ray Diffraction:

The compound of formula 2 in amorphous form and in crystalline form, the hemiethanolate compound of formula 3 and a crystalline solvate with ethanol of the compound 4′-demethylepipodophyllotoxin 2″,3″-bispentafluorophenoxyacetyl-4″,6″-ethylidene-β-D-glucoside 4′-phosphate other than the hemiethanolate obtained above were studied by X-ray powder diffraction analysis.

The device is a Philips PN 1730 equipped with a horizontal CGR goniometer type C with a copper anticathode (λ=1.54051 Angström, I=20 mA and V=40 kV) (FIGS. 1 to 4).

The samples are subjected to the analysis under the following operating conditions:

explored angle range from 3 to 25 degrees θ

acquisition time per point of 500 ms

5 acquisitions (FIGS. 1 to 4).

For FIG. 5, the device is a Philips X'Pert PW 3040 equipped with a goniometer with a copper anticathode (Kα line).

The explored angle range is 2 to 50°2θ.

The counting is 2 or 2.4 s per step, the step having a value of 0.02°2θ.

The sample was measured without special treatment other than the application of a weak pressure so as to obtain a planar surface. A Kapton sheet was placed over the sample so as to prevent contamination of the chamber with the cytostatic sample. The atmosphere used is ambient air.

For FIG. 6, the diagram was recorded on a Philips X'Pert MPD device equipped with an Xcelerator detector and a copper anticathode (K-alpha line, intensity: 20 mA, voltage 40 kV, wavelength lambda: 1.5418367 angström) without a monochromator and with a nickel filter.

The recording was carried out in continuous mode. The scans are in omega 2θ.

The counting is 100 s per step, the value of the step being 0.02°2θ.

The intensity at 100% is attributed to the line with the highest value for the number of counts. The number of counts is proportional to the absolute intensity of each of the lines.

Results:

The diffractogram shows that the crystallinity (related to the number of counts of the line at 100%) is clearly very great in the case of the sample of formula 2 (FIGS. 2 and 6) and also in that of the solvate sample of formula 3 (FIG. 3) and in the other solvate (FIGS. 4 and 5) . The amorphous sample of formula 2 does not show such lines (FIG. 1).

Thermomicroscopy:

A physicochemical thermomicroscopy study was carried out showing a crystalline state with the hemiethanolate of formula 3 with an increase in temperature.

The device used is the FP52 system connected to the FP5 programming device (Mettler). The samples are placed between sliding cover slips and heated according to a programmed rate between 30 and 150° C.

The sample of formula 2 in amorphous form remains amorphous and does not crystallize with an increase in temperature.

The hemiethanolate sample of formula 3 is crystalline. It exhibits a loss of solvent up to the temperature of 150° C., melting occurs at around 165° C. with decomposition.

Stability Study

Demonstration of the Stability in Powdered Form

Studies of stability in powdered form were carried out on the compound of formula 2 in amorphous form and on the compound of formula 3 in crystalline form, obtained above, under the following conditions:

At 40° C. in a closed bottle for 1 month.

At 30° C., at 75% relative humidity, in an open bottle for 1 month.

For the closed-bottle stability studies, the samples are sealed in amber-colored glass vials (Alltech ref. vial 98037, ref. stopper 6687).

The open-bottle stability studies are carried out by storing the powder in a desiccator containing a saline solution saturated with NaCl (75% relative humidity/30° C.).

The content of free phosphate compound of formula 2 is evaluated by internal standardization with the following HPLC chromatographic system:

Column: C₈ Symmetry, 5 μ, 250×4.6 mm (waters)

Eluent: CH₃CN/H₂O/KH₂PO₄ 600/400/3.4 (ml/ml/g) adjusted to pH 4 with 10% H₃PO₄

Flow rate: 1 ml/min

Detection: 220 nm.

The analyses are carried out on the Merck Hitachi Lachrom HPLC system equipped with an L-7100 pump, an L-7200 autosampler, an L-7450 diode array detector and HSM D-7000 system management software.

The results of this study are summarized in table 1 below:

TABLE 1
% of degradation products of the compound of
formula 2 in amorphous form and of the compound of
formula 3 in crystalline form according to the
conservation conditions

Conservation conditions

| | % of degradation | % of degradation |
|---|---|---|
| | products after | products after |
| | 1 month at 40° C. | 1 month at 30° C. |
| | in a closed | with 75% relative |
| Compounds | bottle | humidity |
| Compound of | 11.9% | 50% |
| formula 2 in | | |
| amorphous form | | |
| Compound of | 1.5% | 1.8% |
| formula 3 in | | |
| crystalline form | | |

The studies of stability in powdered form show that the compound of formula 2 in amorphous form is less stable than the hemiethanolate compound of formula 3 in crystalline form, regardless of the stability conditions:

At 40° C. after 1 month in a hermetically closed bottle, 11.9% of degradation products appeared for the compound of formula 2 in amorphous form, against 1.5% for the hemiethanolate compound of formula 3 in crystalline form.

At 30° C. after 1 month in the presence of relative humidity, 50% of degradation products appeared for the compound of formula 2 in amorphous form, against 1.8% for the hemiethanolate compound of formula 3 in crystalline form.

A characteristic of the invention is thus the advantage in terms of stability of the hemiethanolate crystalline form of formula 3 compared with its amorphous free form (formula 2).

Pharmacological Study

Since, at the clinical studies stage, the phosphate form salified with N-methyl-D-glucamine, of formula 1, cannot be used under good conditions, the study of the amorphous free phosphate form of formula 2 was envisaged. It became apparent in the course of the pharmacological study that this compound in its amorphous free phosphate form did not have the in vivo activity demonstrated with its N-methyl-D-glucamine salt, as published in British Journal of Cancer (2000), 83(11), 1516-1524. On the other hand, the crystalline form obtained either in the form of the hemiethanolate of formula 3 or in the free form of formula 2 exhibits the required pharmacological activity.

Materials and Methods

Dissolution of the Compounds for the in vivo Evaluation

The various amorphous and crystalline forms of the compounds of formulae 2 and 3 are dissolved/suspended in a mixture containing 5% of Tween 80 and 95% of serum containing 5% glucose.

Experimental Tumor Model

The model used is the P388 leukemia (Dykes, D. J. and Waud, W. R.: Murine L1210 and P388 Leukemias. In Tumor Models in Cancer Research, Teisher, B. A. ed., Humana Press Inc., Totowa, N.J., pp. 23-40, 2002), which is maintained by successive intraperitoneal transplantations on DBA/2 mice (DBA/2JIco, Charles River), as has been previously described (Kruczynski, A. and Hill, B. T.: Classic in vivo cancer models: Three examples of mouse models used in experimental therapeutics. Current Protocols in Pharmacology Unit 5.24:5.24.1-5.24.16, 2001).

Experimental Chemotherapy

The animals are housed, maintained and handled in accordance with the Guidelines for the Welfare and Use of Laboratory Animals (National Research Council, 1996) and with European directive EEC/86/609, under the direction of the researchers authorized to supervise experiments in animals. Furthermore, all the experiments are carried out in accordance with the French legislation and the rules of the Ethics Committee of the Centre de Recherche Pierre Fabre [PF Research Center], which are based on the directives of the UKCCCR (United Kingdom Coordinating Committee on Cancer Research) for the welfare of animals in experimental oncology (Workman, P., Twentyman, P., Balkwill, F., Balmain, A., Chaplin, D., Double, J., Embleton, J., Newell, D., Raymond, R., Stables, J., Stephens, T., and Wallace, J.,: United Kingdom coordinating committee on cancer research (UKCCCR) guidelines for the welfare of animals in experimental neoplasia. Br. J. Cancer 77: 1-10, 1998). The experiment is carried out according to a protocol already previously described (Kruczynski, A., Colpaert, F., Tarayre, J. P., Mouillard, P., Fahy, J., and Hill, B. T.: Preclinical in vivo antitumor activity of vinflumine, a novel fluorinated Vinca alkaloid. Cancer Chemother. Pharmacol. 41: 437-447, 1998). This consists in implanting 10⁶ P388 leukemia cells, per mouse, in C2DF1 hybrid mice (CD2F1/Cr1BR, Charles River, St Aubin-les-Elbeuf, France), intravenously, on day zero. After random distribution of the animals in the treatment and control cages, the compounds to be evaluated are administered in a single intraperitoneal injection the day after the tumor transplantation, on day 1. The animals are subsequently monitored every day and weighed twice a week, and any clinical reaction is noted.

Evaluation of the Antitumor Activity

Survival is the parameter for evaluating the antitumor activity. The increase in survival is defined by the T/C ratio (%), corresponding to: (Median of survival of the treated group/median of survival of the control group)×100.

Results

Evaluation of the Antitumor Activity of the Various Forms of the Compounds of Formulae 2 and 3 on the P388 Leukemia Model in vivo

The three forms of compounds were evaluated in vivo, i.e.: the compound of formula 2 in amorphous form (amorphous formula 2), the compound of formula 2 in crystalline form, dried in order to remove all traces of the ethanol solvent (crystalline formula 2) and the compound of formula 3 in crystalline form containing half a mole of ethanol (crystalline formula 3). The results are given in table 2 below:

TABLE 2
value of T/C (as %) for the compounds of
formula 2 or 3 in amorphous or crystalline form
according to the dose of compound injected

| T/C values (%): | Dose (mg/kg) | | |
|---|---|---|---|
| Compound | 40 | 80 | 160 |
| Crystalline formula 2 | 143 | 157 | 300 |
| Crystalline formula 3 | 143 | 143 | 329 |
| Amorphous formula 2 | 100 | 114 | 157 |

The results show that the two compounds of formulae 2 and 3 in crystalline form induce a significant increase in survival of the animals starting from the dose of 40 mg/kg, which is reflected by a T/C ratio value of 143%, indicating that the treatment of the animals with one or other of the two compounds in crystalline form has made it possible to prolong the survival of the animals by 43%. In fact, according to the criteria of the NCI (National Cancer Institute), a T/C value is considered to be significant if it is at least greater than 120%, and it is considered to be very highly significant or to reflect a high-level antitumor activity if it is greater than 175% (Venditti, J. M.: Preclinical drug development: Rationale and methods. Semin. Oncol. 8: 349-361, 1981). The antitumor activity induced by the compounds of formula 2 or 3 in crystalline form increases with the dose of compound, to reach an optimum value at the dose of 160 mg/kg, which is reflected by T/C values of 329% for the compound of formula 3 in crystalline form and of 300% for the compound of formula 2 in crystalline form. These values indicate that the treatment of the animals suffering from P388 leukemia with the compounds of formula 2 or 3 in crystalline form made it possible to prolong the survival of the animals by 200% or 229%, respectively.

Unlike the crystalline forms, the compound of formula 2 in amorphous form does not induce any significant antitumor activity at the doses of 40 and 80 mg/kg, which is reflected by T/C values of less than 120%, i.e. 100% and 114%, respectively. The compound of formula 2 in amorphous form induces antitumor activity only at the dose of 160 mg/kg, but this activity is, however, much less than the activity obtained with the compounds of formula 2 or 3 in crystalline form at the same dose. Specifically, the T/C value obtained at the dose of 160 mg/kg for the compound of formula 2 in amorphous form is 157%, whereas it is 329% and 300%, respectively, for the compounds of formulae 3 and 2 in crystalline form.

It should be noted that these activities do not induce any significant weight loss in the animals, suggesting that the compounds are well tolerated by the animal.

In conclusion, the compounds of formula 2 or 3 in crystalline form induce a high-level antitumor activity on the P388 leukemia model, which activity is clearly greater than the activity of the compound of formula 2 in amorphous form.

These compounds therefore provide a major advantage in the therapeutic arsenal against cancer. When administered parenterally, as an infusion, or orally, they make it possible to treat cancers of all types, whether these are liquid tumors or solid tumors. They can be administered to patients as a first-line treatment or after one or more treatments by surgery, radiotherapy or chemotherapy. These compounds can be administered alone or in combination with other chemotherapies.

1. A crystalline compound 4′-epipodophyllotoxin 2″,3″-bispentafluorophenoxyacetyl-4″,6″-ethylidene-β-D-glucoside 4′-phosphate in free form of formula 2 below: 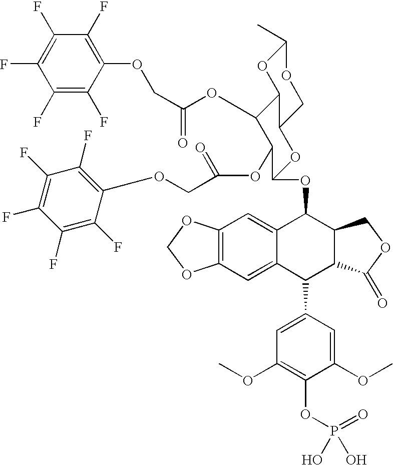
or in the form solvated with ethanol.

2. The crystalline compound as claimed in claim 1, wherein the solvate with ethanol is a hemiethanolate solvate of formula 3 below:

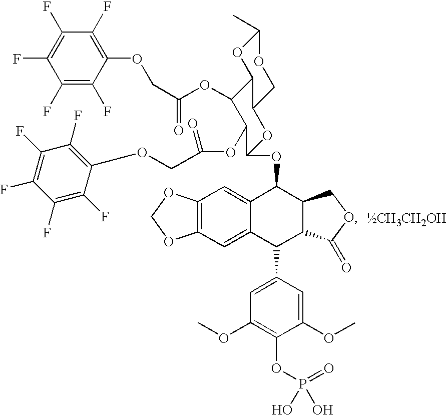

3. The crystalline compound as claimed in claim 1 in free form of formula 2, wherein its X-ray diagram corresponds substantially to that of FIG. 2 or 6.

4. The crystalline compound as claimed in claim 1 or 2 in the form of the hemiethanolate solvate of formula 3, wherein its X-ray diagram corresponds substantially to that of FIG. 3.

5. A method for preparing the crystalline compound as claimed in any of claims 1 or 2 in the form of the hemiethanolate solvate, wherein the compound of formula 2 in amorphous form is dissolved in anhydrous ethanol in the presence of ultrasound.

6. The method as claimed in claim 5, wherein the ultrasound is applied for at least 20 minutes.

7. A method for preparing the crystalline compound as claimed in claim 1 in free form, wherein the crystalline compound in the form of the hemiethanolate solvate of formula 3 is dried.

8. A method for preparing the crystalline compound as claimed in any of claims 1 or 2 in the form solvated with ethanol, it comprises the following successive steps:
a) addition of ethanol to the compound of formula 2 in amorphous form,
b) concentration of the solution,
c) crystallization of the 4′-demethylepipodophyllotoxin 2″,3″-bispentafluorophenoxyacetyl-4″,6″-ethylidene-β-D-glucoside 4′-phosphate in the form solvated with ethanol, by cooling the concentrated solution at 10° C. for 1.5 h and adding an initiator consisting of a crystal of the compound of formula 2 in free form or of the compound of formula 3 in the form of the hemiethanolate solvate,
d) filtration at 10° C. and rinsing with ethanol,
e) drying of the product obtained.

9. A medicinal product comprising a crystalline compound as claimed in any one of claims 1 or 2.

10. A method for anticancer treatment for liquid tumors or solid tumors comprising the administration of an effective amount of the medicinal product as claimed in claim 9, in a patient in need thereof.

11. A method for increasing the therapeutic efficacy of topoisomerase 1 and 2 inhibitor compounds, for the treatment of tumors that are refractory to the usual therapies comprising the administration of an effective amount of the medicinal product as claimed in claim 9.

12. The medicinal product as claimed in claims 9, wherein it is in a form intended for oral or parenteral administration.

13. The medicinal product as claimed in of claims 9, wherein it also comprises another anticancer agent.

14. The method of claim 7, wherein the hemiethanolate solvate of formula 3 is dried under vacuum.

15. The method of claim 10, wherein the tumors are small cell lung cancer, embryonic tumors, neuroblastomas, kidney cancer, pediatric tumors, Hodgkins and nonhodgkins lymphomas, acute leukemias, placental choriocarcinomas, and mammary adenocarcinomas.

16. The method of claim 11, wherein the tumors are colorectal cancers, melanomas, gliomas, prostate cancer and breast cancer.
